(19) United States
(12) Patent Application Publication
(10) Pub. No.: US 2023/0345979 A1
(43) Pub. Date: Nov. 2, 2023

(54) METHOD FOR PREPARING CULTURED MEAT ON BASIS OF CELL COATING TECHNIQUE, AND CULTURED MEAT PREPARED THEREBY

(71) Applicant: Industry-Academic Cooperation Foundation, Yonsei University, Seoul (KR)

(72) Inventors: Jinkee HONG, Seoul (KR); Sohyeon PARK, Seoul (KR)

(73) Assignee: Industry-Academic Cooperation Foundation, Yonsei University, Seoul (KR)

(21) Appl. No.: 18/006,447

(22) PCT Filed: Jul. 22, 2021
(86) PCT No.: PCT/KR2021/009504

§ 371 Date: Jan. 23, 2023

(30) Foreign Application Priority Data
Jul. 22, 2020 (KR) 10-2020-0090734

(51) Int. Cl. C12N 5/077; A23L 29/275; A23L 13/00; A23L 29/262

(52) U.S. Cl. CPC A23L 13/03; A23L 29/262; A23L 29/275; C12N 5/0658; C12N 2513/00; C12N 2533/72

(57) ABSTRACT

The present disclosure provides a method for producing cultured meat, the method including: coating surfaces of cells usable for producing cultured meat to form a nanofilm; culturing the coated cells; inducing proliferation of the cultured cells; and allowing muscle tissue to be formed from the differentiated cells, and cultured meat produced using the same. As cell protection and cell adhesion are increased, the cell proliferation and differentiation efficiency is increased, such that an environment optimized for cultured meat production may be created through mass proliferation of the cells.

### CROSS-REFERENCE TO RELATED APPLICATIONS

This application is a 35 U.S.C. §371 national phase of PCT International Application No. PCT/KR2021/009504, filed Jul. 22, 2021, which claims the benefit of priority under 35 U.S.C. §119 to Republic of Korea Patent Application No. 10-2020-0090734, filed Jul. 22, 2020, the contents of which are incorporated herein by reference in its entirety.

### FIELD

The present disclosure relates to a method for producing cultured meat based on a cell coating technique and cultured meat produced using the same.

### BACKGROUND

According to the report of the Food and Agriculture Organization (FAO), the world population is expected to increase from 7.6 billion in 2018 to 9.5 billion in 2050. However, while the yield of crops is expected to decrease due to the earth’s abnormal climate such as global warming, the demand for grains for feed is expected to increase, which will lead to an increase in production costs and a reduction in production area. Therefore, livestock products as food resources are expected to become expensive.

Although animal-derived foods such as livestock products are expensive in terms of energy and production, the animal-derived foods have directly contributed to securing human nutrition because they are the best source of high-quality proteins and micronutrients essential for normal growth and health of humans. In particular, essential amino acids are nutrients that must be taken with foods because they are not synthesized in the body or are synthesized in significantly small quantities. Considering the prediction that the demand for animal-derived foods will reach 550 million tons in 2050, doubling the current level, there is a limit to handling proteins required to supply the essential amino acids with traditional livestock production methods.

Meanwhile, as a solution to a meat shortage problem in the future, cultured meat has recently been attracting attention. The cultured meat, also called substitute meat, refers to edible meat that is obtained through cell proliferation using cell engineering by culturing cells of living animals in a laboratory without going through a process of raising livestock. The cultured meat is also called “in vitro meat” or “lab-grown meat” in the sense that it is grown in a test tube, “artificial meat” in the sense that it is synthesized using stem cells by humans rather than in nature, “clean meat” in the sense that it is produced in a clean production facility rather than in a traditional breeding facility, or “bioartificial muscles (BAMs)” in the sense that muscle fibers constituting cultured meat are cultured.

The idea of the cultured meat was raised quite a long time ago, and in 1932, British Prime Minister Winston Churchill wrote in a book called “Fifty Years Hence” that fifty years hence, we shall escape the absurdity of growing a whole chicken in order to eat the breast or wing by growing these parts separately under a suitable medium. Later, in 1999, Dr. Willem van Eelen of the University of Amsterdam in the Netherlands, who is called the godfather of cultured meat, obtained an international patent for the theory on cultured meat, and in 2002, succeeded in culturing muscle tissue derived from goldfish in a petri dish in the laboratory.

The method for producing cultured meat that is currently mainly used is as follows. A tissue is collected from a living animal, and then stem cells are isolated from the tissue. Thereafter, the isolated stem cells are cultured into myocytes in the laboratory, grown for several weeks, and then subjected to muscle fiber coloring and fat mixing, thereby producing cultured meat. In this case, as a culture method, scaffolds are used, and self-organizing method techniques are also used.

Although the idea of cultured meat began quite a long time ago, animals still need to be slaughtered in a process of collecting tissues from living animals and isolating stem cells from the tissues to produce cultured meat. In addition, the current cultured meat production technique takes a long time to culture stem cells collected from living cells into myocytes, which still keeps the production cost of cultured meat at a high level. Furthermore, the efficiency of proliferation and differentiation of cells is reduced during long-term culture, and as a result, a yield of cells of cultured meat is significantly reduced.

This is a major technical problem that hinders commercialization and mass production of cultured meat. In order to commercialize the cultured meat technique, there are demands for developing a tissue culture technique and securing a mass production technique.

### Related Art Document

### Patent Document

U.S. Pat. No. 7,270,829 (Sep. 18, 2007)

### SUMMARY

The present disclosure has been made to solve the above technical problem, and an object of the present disclosure is to provide a method for producing cultured meat that may induce proliferation and differentiation of a large amount of cells with minimal stem cells obtained from living animals.

Further, another object of the present disclosure is to provide a method for producing cultured meat that maintains adhesion of cells and maintains high cell proliferation and differentiation efficiency even during long-term culture of cells of cultured meat.

Further, still another object of the present disclosure is to provide a method for producing cultured meat that may control cell behavior by protecting cells and continuously applying a stimulus to cells during a cell culture process.

Still another object of the present disclosure is to create an environment optimized for mass proliferation and differentiation of cells for cultured meat production by controlling cell protective effects and cell properties by coating.

Still another object of the present disclosure is to provide cost-effective cultured meat that may be produced quickly at low cost according to high cell proliferation and differentiation efficiency.

In one general aspect, a method for producing cultured meat includes: coating surfaces of cells usable for producing cultured meat to form a nanofilm; culturing the coated cells; inducing proliferation of the cultured cells; and allowing muscle tissue to be formed from the differentiated cells.

In the method for producing cultured meat according to an aspect of the present disclosure, the cells usable for producing cultured meat may be mesenchymal stem cells (MSCs), induced pluripotent stem cells (iPSCs), satellite cells, adipocytes, or embryonic stem cells.

In the method for producing cultured meat according to an aspect of the present disclosure, the coating may be performed to form a multilayer nanofilm using one or two or more selected from the group consisting of electrostatic attraction, van der Waals force, hydrophobic bonding, hydrogen bonding, and covalent bonding.

In the method for producing cultured meat according to an aspect of the present disclosure, the nanofilm may be formed by alternately stacking a positively charged material and a negatively charged material.

In the method for producing cultured meat according to an aspect of the present disclosure, the positively charged material may be one or two or more selected from the group consisting of chitosan, starch, collagen, gelatin, fibrinogen, silk fibroin, casein, elastin, laminin, and fibronectin.

In the method for producing cultured meat according to an aspect of the present disclosure, the negatively charged material may be one or two or more selected from the group consisting of hyaluronic acid, alginate, pectin, tannic acid, lignin, cellulose, heparin, gellan gum, ester gum, carrageenan, agar, xanthan gum, gum arabic, glucomannan, carboxymethylcellulose gum (CMC), guar gum, locust bean gum, tamarind gum, and tara gum.

In the method for producing cultured meat according to an aspect of the present disclosure, a thickness of the nanofilm may be 50 to 5,000 nm.

In the method for producing cultured meat according to an aspect of the present disclosure, the coated cells may be cultured on a scaffold or in a bioreactor.

In the method for producing cultured meat according to an aspect of the present disclosure, in the culturing of the coated cells, the coated cells may be stimulated by an ultrasonic wave, an electric current, an electromagnetic field, a magnetic field, or a combination thereof.

In the method for producing cultured meat according to an aspect of the present disclosure, the method may further include adding a fat and a colorant to the muscle tissue.

In another general aspect, there is provided cultured meat produced by the method for producing cultured meat.

The cultured meat according to an aspect of the present disclosure may be a substitute for chicken, pork, beef, goat, lamb, duck, or fish.

In the method for producing cultured meat according to the present disclosure, the cells are protected from external stress through coating of the surfaces of the cells, and stable cell proliferation is performed, such that an environment optimized for cultured meat production may be created.

In the method for producing cultured meat according to the present disclosure, cell membrane proteins such as cadherins, which are involved in cell-to-cell interactions, may be stabilized to enhance adhesion between cells, which enhances the ability to induce differentiation into myocytes.

In the method for producing cultured meat according to the present disclosure, as a stimulus is continuously applied to the cells, the cell behavior may be controlled, such that myocytes may be obtained with a high yield through mass proliferation and differentiation.

The present disclosure provides cost-effective cultured meat quickly produced at low cost according to high cell proliferation and differentiation efficiency.

### BRIEF DESCRIPTION OF THE DRAWINGS

FIG. 1 illustrates a culture process in a method for producing cultured meat according to Comparative Example 1 and Example 1 of the present disclosure, and is a comparative schematic view showing proliferation results according to the presence or absence of coating of single cells.

FIG. 2 is a schematic view showing a process of coating cells by alternately stacking a positively charged layer and a negatively charged layer.

FIG. 3A is a schematic view briefly showing a production process of a crosslinked porous nanofilm (X-linked (CHI/CMC)) according to Example 1-2 of the present disclosure.

FIG. 3B is a schematic view briefly showing a production process of a cell culture platform for producing cultured meat according to Example 1-3 of the present disclosure.

FIG. 4A is a graph showing results of FT-IR spectral analysis of a crosslinked porous nanofilm (X-linked (CHI/CMC)) according to Experimental Example 1-1 of the present disclosure.

FIG. 4B shows results of comparative analysis of AFM images of a crosslinked porous nanofilm (X-linked (CHI/CMC)) and a non-crosslinked porous nanofilm according to Experimental Example 1-2 of the present disclosure.

FIG. 5A illustrates a state of a film in which C-phycocyanin is incorporated into a porous structure thereof and a confocal microscope image thereof, in which in the case of the crosslinked film, a clear fluorescence image is observed after incorporation of C-phycocyanin.

FIG. 5B illustrates SEM images of the non-crosslinked film, the crosslinked film, and the film into which C-PC is incorporated.

FIG. 6A illustrates confocal microscope images of human fibroblasts (HDFs) coated with a 1 bilayer, a 2 bilayer, and a 3 bilayer according to an embodiment of the present disclosure.

FIG. 6B is a graph showing a comparison of cell proliferation results of a case where culture is performed by coating human fibroblasts with a 3 bilayer and a case where culture is performed without coating human fibroblasts according to an embodiment of the present disclosure.

FIG. 7 is a graph showing results of cell proliferation according to Experimental Example 2 of the present disclosure.

FIG. 8 is a graph showing a comparison of C-phycocyanin release properties in a film with a protective layer and a film without a protective layer.

FIG. 9 is an optical microscope image showing a cell proliferation state of each group according to Experimental Example 2 of the present disclosure.

FIG. 10 is an image showing states of cultured meat produced according to Example 2 of the present disclosure before and after cooking.

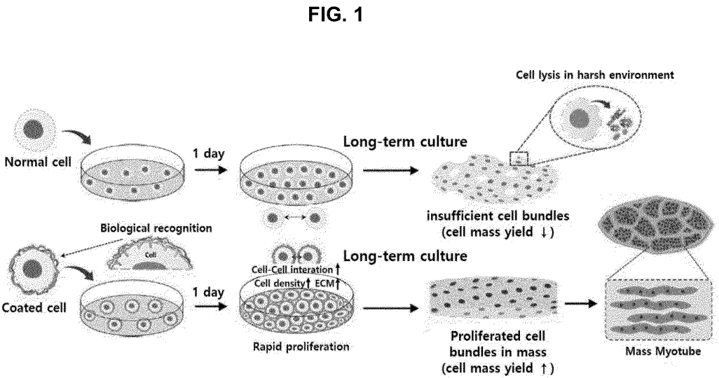

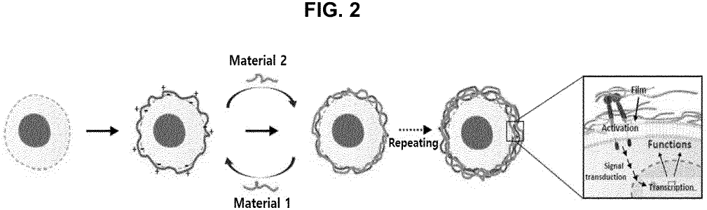

### DETAILED DESCRIPTION

Hereinafter, a method for producing cultured meat according to the present disclosure and cultured meat produced using the same will be described in detail. Here, unless otherwise defined, all the technical terms and scientific terms have the same meanings as commonly understood by those skilled in the art to which the present disclosure pertains. The terms used in the description of the present disclosure are only for effectively describing a certain example rather than limiting the present disclosure.

In addition, a description of the known effects and configurations unnecessarily obscuring the gist of the present disclosure will be omitted in the following description. Hereinafter, a unit used in the present specification without special mention is based on weight, and as an example, a unit of % or a ratio refers to wt% or a weight ratio.

In addition, terms “first”, “second”, A, B, (a), (b), and the like may be used in describing components of the present disclosure. These terms are used only in order to distinguish any component from other components, and features, sequences, or the like of corresponding components are not limited by these terms.

In addition, unless the context clearly indicates otherwise, singular forms used in the specification of the present disclosure may be intended to include plural forms.

Hereinafter, a method for producing cultured meat according to the present disclosure will be described in detail.

First, in a generally known method for producing cultured meat, a tissue is collected from a living animal, muscle satellite cells are extracted and separated from the tissue, and the prepared cells are placed in a bioreactor to allow the cells to proliferate. Then, the proliferated cells may be transferred to a scaffold or self-organized and differentiate into muscle tissue in a differentiation medium. However, such a process is not easy to commercialize due to high production costs because the actual process is complicated and expensive nutrients should be continuously supplied for cell growth.

Accordingly, the present disclosure is to provide a method for producing cultured meat that includes a simple process capable reducing production costs due to a cell growth medium and implementing stable mass proliferation of cells.

A method for producing cultured meat according to the present disclosure includes: coating surfaces of cells usable for producing cultured meat to form a nanofilm; culturing the coated cells; inducing proliferation of the cultured cells; and allowing muscle tissue to be formed from the differentiated cells. Examples of the cells usable for producing cultured meat include mesenchymal stem cells (MSCs), induced pluripotent stem cells (iPSCs), satellite cells, adipose-derived stem cells (ASCs), and embryonic stem cells.

Each step will be described in more detail below.

The step of isolating stem cells usable for producing cultured meat and coating surfaces of the stem cells to form a nanofilm may be performed by alternately treating a solution containing a positively charged material and a solution negatively charged material so as to alternately stack a positively charged material layer and a negatively charged material layer. The coating may be performed using one or two or more selected from the group consisting of electrostatic attraction, van der Waals force, hydrophobic bonding, hydrogen bonding, and covalent bonding, thereby forming a multilayer nanofilm.

Specifically, the method includes alternately immersing the cells usable for producing cultured meat in two oppositely charged coating solutions including a first coating solution containing a positively charged material and a second coating solution containing a negatively charged material. That is, the cells are immersed in the first coating solution to introduce a positively charged layer into cell membrane surfaces, and then the cells are immersed in the second coating solution to stack a negatively charged layer on the positively charged layer, such that a layer-by-layer (LbL) assembly is performed on the surfaces of the cells, and a multilayer nanofilm may be formed by repeating this operation n times.

The oppositely charged layers obtained by alternate bonding of the positively charged layer and the negatively charged layer may stably maintain bonding through electrostatic attraction, and such a multilayer nanofilm may protect the cells in a stable state for a long period of time.

In an embodiment of the present disclosure, the first coating solution may be prepared by adding 10% fetal bovine serum (FBS) and a positively charged material to Dulbeco’s Modified Eagle’s Media (DMEM), and the second coating solution may be prepared by adding 10% FBS and a negatively charged material to DMEM. In this case, the positively charged material or the negatively charged material may be contained at a concentration of 0.01 to 10 mg/ml, preferably 0.1 to 5 mg/ml, and still more preferably 0.5 to 3 mg/ml.

The first coating solution or the second coating solution may further contain a plurality of cell growth factors required for cell culture, for example, an epidermal growth factor (EGF), an insulin like growth factor (IGF-1), a platelet-derived growth factor (PDGF), a transforming growth factor-beta (TGF-β), a vascular endothelial growth factor (VEGF), a leukemia inhibitory factor (LIF), a basic fibroblast growth factor (bFgF), and the like. However, in addition to DMEM, α-MEM Eagles’s MEM, Iscove’s MEM, 199 medium, CMRL 1066, RPMI 1640, F12, F10, Way-mouth’s MB752/1, or McCoy’s 5A may be used as the medium.

In addition, the medium is not limited to FBS, and may be a serum medium to which bovine calf serum (BCS) or horse serum is added, or a serum-free medium containing additives.

Alternatively, the medium may be a serum replacement medium containing nutrients capable of replacing animal-derived serum.

Specifically, the nutrients capable of replacing animal-derived serum may be, for example, active ingredients derived from microalgae. More specifically, the nutrient may be C-phycocyanin. C-phycocyanin is an active ingredient extracted from cyanobacteria with a multicellular filamentous form called Spirulina platensis, and is known to have beneficial functions such as antioxidant and anti-inflammatory effects and enhancement of immune function. When the C-phycocyanin is contained as a nutrient for enhancing cell proliferation, it is cost-effective because the use of animal-derived serum may be significantly reduced, and as cell proliferation and differentiation of bone marrow hematopoietic cells are enhanced, an improved cell proliferation effect may be provided.

Since the constituting ingredients of the culture solution may be adjusted as needed, it is not limited to the composition described above.

In an embodiment of the present disclosure, a thickness of the nanofilm may be 5 to 5,000 nm. The thickness of the nanofilm may be controlled according to a desired application, and it is preferable that the thickness of the nanofilm is within the above range so as not to act as a barrier to material diffusion as a dense layer is formed on the cells. The thickness of the nanofilm is preferably 10 to 4,000 nm, and more preferably 20 to 2,000 nm, in terms of maintaining the cell performance. The nanofilm may have two or more layers (one or more bilayers), and preferably, may have 4 to 40 layers. More preferably, the nanofilm may have 10 to 30 layers.

In an embodiment of the present disclosure, if necessary, a cleaning process may be further included between introduction of the positively charged layer and then introduction of the next negatively charged layer, or between introduction of the negatively charged layer and then introduction of the next positively charged layer, within a range that does not impair achievement of the object of the present disclosure. The cleaning process refers to a step for removing the materials stacked on the surfaces of the cells or the charged layer with weak bonding, and may be performed using the same solvent as that of the first coating solution or the second coating solution. As a specific example, a solution obtained by adding 10% FBS to DMEM may be used as a cleaning solution. Through the cleaning process, an effect of uniformly and rapidly forming a coating layer on the surface of the cell is achieved.

The positively charged material and the negatively charged material should be edible for the production of cultured meat, and are preferably biocompatible organic polymers or inorganic materials.

In an embodiment of the present disclosure, as a specific example, the organic polymer may be one or two or more selected from the group consisting of chitosan, chitin, starch, collagen, gelatin, fibrinogen, silk fibroin, casein, elastin, laminin, and fibronectin. Preferably, the organic polymer may be chitosan, collagen, gelatin, or laminin, but is not particularly limited thereto, as long as it is a cationic polysaccharide polymer. The negatively charged material may be one or two or more selected from the group consisting of hyaluronic acid, alginate, pectin, tannic acid, lignin, cellulose, heparin, gellan gum, ester gum, carrageenan, agar, xanthan gum, gum arabic, glucomannan, carboxymethylcellulose gum (CMC), guar gum, locust bean gum, tamarind gum, and tara gum. Preferably, the negatively charged material may be carboxymethylcellulose gum (CMC), carrageenan, xanthan gum, or agar, but is not particularly limited thereto, as long as it is an anionic polysaccharide polymer.

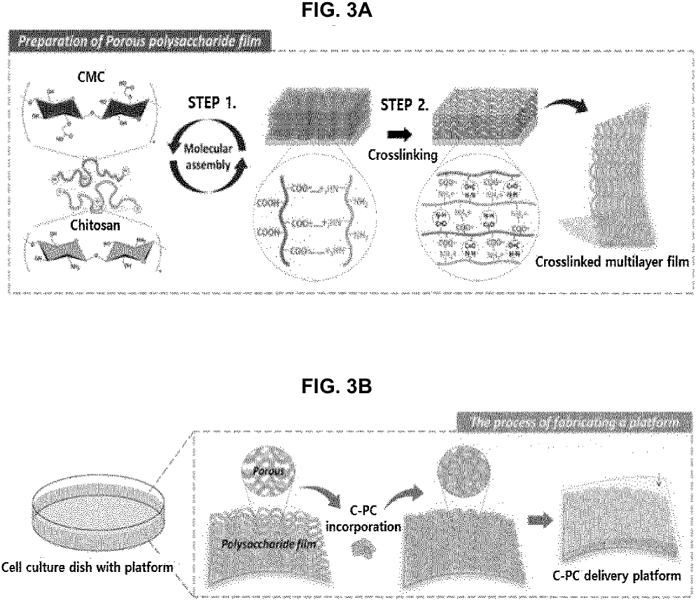

Referring to FIG. 3A, the forming of the nanofilm will be described in detail. Chitosan, which is a positively charged polysaccharide, includes a plurality of NH₂ functional groups and is positively charged because it is converted into NH₃⁺ in an aqueous solution with a pH of 4 to 5. CMC, which is a negatively charged polysaccharide, includes a plurality of COOH functional groups, and is negatively charged because it is converted into COO⁻ in an aqueous solution with a pH of 4 to 5. Accordingly, a chitosan layer and a CMC layer may be assembled into LbL according to the electrostatic interaction to form a multilayer nanofilm.

As a preferred embodiment of the present disclosure, the method may further include forming crosslinking in the multilayer nanofilm. The crosslinking may be induced by a crosslinking agent, and a representative example of the crosslinking agent may be ethyl(dimethylaminopropyl)carbodiimide (EDC)/hydroxysuccinimide (NHS). A first crosslinking may be performed by forming a stable amide bond between an ester group of CMC and an amine group of chitosan using the EDC/NHS principle. Alternatively, a reactive end of glutaraldehyde may be induced to form a covalent bond between a hydroxyl group and a primary amine group of the polysaccharide by using glutaraldehyde, thereby further performing a second crosslinking between the polysaccharide chains. The crosslinked film exhibits a rough structure with multiple pores, and in this case, polymer loading and release behavior of the cell growth factor and the like may further actively occur.

The crosslinking may effectively act to incorporate and immobilize the cell growth factor in the porous film. Specifically, when the cell growth factor is negatively charged, the cell growth factor may electrostatically interact with an amine group in the porous film, and a functional group in the cell growth factor and various functional groups of the polysaccharide in the film may form hydrogen bonds. Alternatively, the cell growth factor may be further immobilized in the film by reacting with the reactive end of the crosslinking agent.

In the present disclosure, after the forming of the nanofilm on the surfaces of the cells, a protective layer may be further coated to induce continuous release of the cell growth factor. The protective layer may be coated on the surface of the nanofilm to reduce the mobility of the cell growth factor so that the cell growth factor incorporated into the nanofilm may be sustainably released.

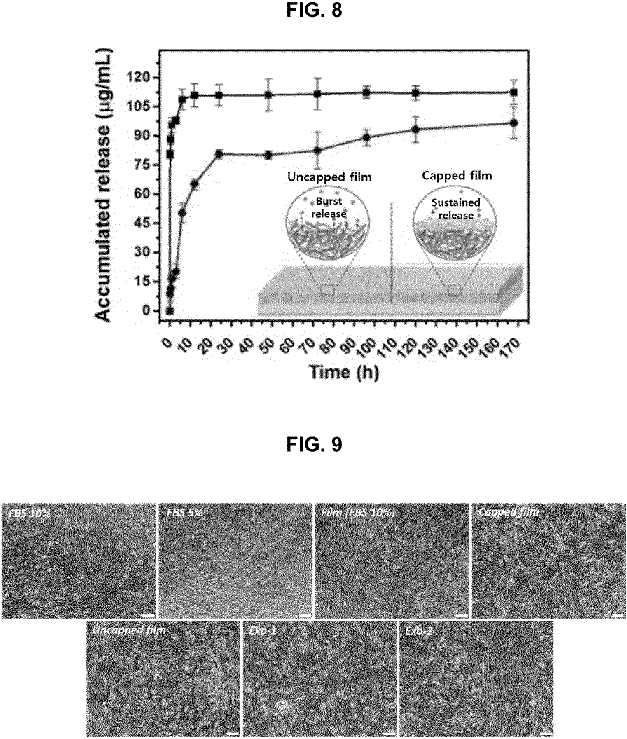

FIG. 8 illustrates a release profile of C-phycocyanin selected as anutrient for enhancing cell proliferation. In the case of a film with a protective layer (capped film) than in the case of a film without a protective layer (uncapped film), the initial rapid diffusion was inhibited, and the sustained release behavior of C-PC was observed. The protective layer is not particularly limited, and the protective layer is preferably a sugar compound in order to increase the stability of the cell nutrient. As a non-limiting example, the protective layer may contain agarose.

In addition, in an embodiment of the present disclosure, the method may further include, after the forming of the nanofilm on the surfaces of the cells, adding a cell growth factor. The cell growth factor may be incorporated inside the porous nanofilm and may be sustainably released. That is, the cell growth factor is immobilized by the electrostatic interaction or crosslinking between the positively charged material and the negatively charged material inside the porous nanofilm. In the case where the organic polymer-based nanofilm is formed on the surfaces of the cells as described above, it is possible to effectively perform functions of protecting the cells from the external environment and controlling the cell behavior by continuously applying a stimulus to the cells.

The stem cells usable for producing cultured meat are sequentially induced to proliferate and differentiate into myoblasts and myocytes, and the myocytes form muscle tissue. Since the quality of meat is formed by muscle movement, it is required to realize muscle tissue similar to that of a living animal. To this end, a method of continuously applying a physical stimulus to muscle fibers may be performed. In particular, in a case where the surfaces of the cells are coated with a polymer material related to an extracellular matrix (ECM), a continuous physical stimulus may be easily applied to the cells, and production of proteins of the muscle fibers may be controlled. Through repeated pulling and loosening of the muscle fibers, production of collagen may be increased or decreased.

In an embodiment of the present disclosure, the inorganic material may be introduced, if necessary, in terms of imparting high strength to the cells. As a specific example, the inorganic material may be calcium phosphate (Ca₃(PO₄)₂), calcium carbonate (CaCO₃), sodium chloride (NaCl), potassium chloride (KCI), magnesium sulfate (MgSO₄), magnesium chloride (MgCl₂), and sodium bicarbonate (NaHCO₃), calcium chloride (CaCl₂), or potassium dihydrogen phosphate (KH₂PO₄), but is not particularly limited thereto, and any inorganic material may be used as long as it corresponds to a biomineral. As a specific example of the present disclosure, in a case where the negatively charged layer is stacked and then the inorganic material is coated, crystallization on the surface may be smoothly performed, which is preferable. In this case, mechanical properties may be supplemented, such that the effect of protecting the cells may be significantly excellent, and cell division may be controlled. When mass proliferation is induced, cell division may be controlled by decomposing the inorganic material by treatment with an acid.

The culture type may be a common two-dimensional or three-dimensional culture known in the art. However, three-dimensional culture may be preferable to realize a tissue similar to an actual biological tissue by a cell-to-cell interaction. Specific examples of the three-dimensional culture include a 3D porous scaffold, a scaffold-free platform through cells themselves or cell sheet techniques, a method of placing cells in a microchip, a method using a hydrogel, and a method using a bioreactor.

The coated cells according to an embodiment of the present disclosure may be cultured on a scaffold or in a bioreactor. A bioreactor is a cylindrical chamber in which factors such as perfusion, temperature, humidity, and gas exchange are logically controlled, and cells may be placed on a scaffold inside the bioreactor to facilitate 3D culture. A scaffold, which is a three-dimensional support, mimics various roles of an extracellular matrix of a living body in a given environment, is involved in adhesion, proliferation, and differentiation of cells, and is ultimately incorporated into a tissue. Since the scaffold is generally composed of a hydrogel, the scaffold may be physically weak, but may provide a biological environment for cells.

Specifically, the coated stem cells differentiate into myoblasts, and the myoblasts are seeded on a scaffold or in a bioreactor to proliferate and differentiate into myocytes. The myoblasts may grow while attached to the scaffold or may grow in the bioreactor by self-organization.

In a preferred embodiment of the present disclosure, the coated stem cells may be seeded and cultured on the scaffold. The coated stem cells may be seeded on a fixed scaffold, or the coated stem cells and a scaffold material may be first mixed, and then the preparation of the scaffold and proliferation of the cells may be simultaneously performed by 3D printing. The stem cells coated according to the present disclosure exhibit an excellent effect of protecting the cells from a physical stimulus applied to the cells by the 3D printing.

In order to produce cultured meat for hamburger patties, sausages, and minced meat, which are mainly used without bones, the stem cells may be seeded and cultured on the scaffold. Alternatively, in order to obtain structured meat such as steak, it may be advantageous for the stem cells to undergo growth by self-organization. The self-organization refers to production of highly organized muscle tissue and cultured meat from stem cells by itself, or production of cultured meat by proliferating existing muscle tissue in a culture medium.

The myoblasts cultured on the scaffold or in the bioreactor differentiate into myocytes, and the myocytes grow into muscle tissue. This process may include a step of allowing the cells to be stimulated by an ultrasonic wave, an electric current, an electromagnetic field, a magnetic field, or a combination thereof. The stimulus is a physical stimulus including a mechanical stimulus or an electrical stimulus, and an appropriate physical stimulus is applied, such that an environment similar to that in an actual body in which various stimuli such as circulatory system, nervous system, muscles, and the like exist may be created. Through this, growth promotion is induced during cell culture, and the shape, function, and development of myocytes may be controlled.

In an embodiment of the present disclosure, the method may further include adding a fat and a colorant to the muscle tissue. The fat may be added in a co-culture method by injecting separately cultured adipocytes into muscle tissue or injecting adipocytes in the process of proliferation of myocytes.

As a specific example of the co-culture, pre-adipocytes and myocytes are uniformly mixed with a scaffold material such as gelatin or collagen, and the mixture is added to a medium to prepare a mixed culture solution. The mixed culture solution is floated layer-by-layer using a 3D cell-printing system, and then proliferation and differentiation of adipocytes and myocytes may be induced. The coated stem cells according to the present disclosure may be stably protected despite external stimuli and may maintain excellent cell adhesion, such that mass proliferation and differentiation are induced. In a case where adipocytes and myocytes are co-cultured, an additional fat addition process is unnecessary, and a tissue similar to actual muscle tissue may be formed according to the interaction between the adipocytes and the myocytes, which may further improve the taste of meat.

In addition, the fat may be added by injecting separately cultured adipocytes into the muscle tissue or adding and mixing a liquid fat with the muscle tissue when the muscle tissue is produced as a patty. This is considered one of the advantages of cultured meat because saturated fatty acids contained in meat may be replaced with beneficial fats. Since the taste of meat is derived from fat between the muscles, in order to realize the taste of cultured meat close to the taste of actual meat, vegetable fat and oils such as soybean oil, corn oil, canola oil, rice bran oil, sesame oil, extracted sesame oil, perilla oil, extracted perilla oil, safflower oil, sunflower oil, cottonseed oil, peanut oil, olive oil, palm oil, coconut oil, and red pepper seed oil, animal fats and oils such as edible beef tallow, edible lard, raw beef tallow, raw pork fat, and fish oil, and edible oil and fat processed products such as blended edible oil, flavored oil, processed oil, shortening, margarine, imitation cheese, and vegetable cream may be used.

The colorant refers to a compound that imparts color to food. In order to reproduce the red color of beef or pork, an artificial colorant, a natural colorant, natural extract (for example, beet root extract, pomegranate extract, cherry extract, carrot extract, red cabbage extract, or red seaweed extract), modified natural extract, natural juice (for example, beet root juice, pomegranate juice, cherry juice, carrot juice, red cabbage juice, or red seaweed juice), modified natural juice, food drug & cosmetics (FD&C) Red No. 3 (erythrosine), FD&C Green No. 3 (fast green FCF), FD&C Red No. 40 (allura red (AC)), FD&C Yellow No. 5 (tartazine), FD&C Yellow No. 6 (sunset yellow FCF), FD&C BLUE No. 1 (brilliant blue FCF), FD&C BLUE No. 2 (indigotine), titanium oxide, annatto, anthocyanin, betanin, β-APE 8 carotenal, β-carotene, black currant, burnt sugar, canthaxanthin, caramel, carmine/carminic acid, cochineal extract, curcumin, lutein, carotenoid, monascin, paprika, riboflavin, saffron, turmeric, and a combination thereof may be used, but are not particularly limited thereto. Additionally, a color developing agent such as nitrite, and ascorbic acid, erythorbic acid, or a salt thereof, which promotes color development of the nitrite, may be further added as a color development aid.

In addition, additionally, in order to prevent rancidity of fat, change in color, or separation of fat, an antioxidant, emulsifying salts, and the like for protein stabilization may be added. The antioxidant, the emulsifying salts, and the like may be used without limitation as long as they are widely used in the art.

In addition, the present disclosure provides cultured meat produced according to the method for producing cultured meat described above. In this case, the cultured meat may be a substitute for chicken, pork, beef, goat, lamb, duck, or fish.

Hereinafter, the method for producing cultured meat according to the present disclosure will be described in more detail with reference to Examples. However, the following Examples are only reference examples for describing the present disclosure in detail, and the present disclosure is not limited thereto and may be implemented in various forms.

### [Preparation Example 1] Production of Cell Culture Platform for Producing Cultured Meat

### 1-1. Production of Porous Nanofilm

A chitosan aqueous solution (CHI, medium Mw, deacetylation = 75 to 85%, Sigma-Aldrich) at a concentration of 1 mg/ml and a carboxymethylcellulose sodium salt aqueous solution (CMC, Mw ≈ 250,000, Sigma-Aldrich) at a concentration of 1 mg/ml were prepared, and the pHs of both solutions were adjusted to 4 using 1 M HCl and NaOH. Oxygen plasma-treated substrates (silicon wafer, slide glass, and OHP film) were immersed in a CHI solution for 10 minutes, and the substrates were cleaned twice with deionized water (DI water) to form a stable positively charged layer on surfaces of the substrates. Subsequently, the positively charged substrates were immersed in a negatively charged CMC solution for 10 minutes, and the substrates were cleaned in the same manner. In this process, a single bilayer (BL) film was formed on the surface of each of the substrates by the electrostatic interaction between CHI and CMC. This alternating deposition was repeated n times to produce a (CHI/CMC) film composed of n BLs.

### 1-2. Production of Crosslinked Porous Nanofilm

After LbL assembly, a crosslinking reaction was introduced twice to obtain a porous internal structure of a film. 1-Ethyl-3-(3-dimethylaminopropyl)carbodiimide hydrochloride (EDC, Mw ≈ 191.71, Daejung)/N-hydroxysulfosuccinimide (NHS, Mw ≈ 115.09, Sigma-Aldrich) was used for a first crosslinking. A substrate coated with the (CHI/CMC) film was immersed in 0.1 M EDC and a 0.05 M solution of 2-(N-morpholino)ethanesulfonic acid hydrate (MES buffer, Mw ≈ 195.2, Sigma-Aldrich) supplemented with 2.5 mM NHS for 20 minutes, and then the substrate was immersed in phosphate buffered saline (1X PBS Gibco® Life Technologies) and deionized water to remove unreacted residues. For a secondary crosslinking, the substrate in which the first crosslinking was completed was incubated in a 2.5% glutaraldehyde solution (Mw ≈ 25,000, Sigma-Aldrich) for 30 minutes, and then the substrate was thoroughly cleaned with deionized water, thereby completing a crosslinked porous nanofilm (X-linked (CHI/CMC)).

### 1-3. Production of Cell Culture Platform for Producing Cultured Meat

A C-phycocyanin (C-PC) solution was prepared at a concentration of 0.5 mg/mL using 1X PBS as a solvent. A substrate coated with the crosslinked porous nanofilm was incubated in the C-PC solution for 12 hours at room temperature in a light-blocking environment so that C-PC was sufficiently incorporated into the film. While the film was dried, agarose was dissolved in deionized water at a concentration of 0.1 w/v%. In order to form a C-phycocyanin protective layer, an agarose solution was applied to the dried film at 25 µl per cm². The prepared film was cured at 4° C. to complete a cell culture platform (capped (CHI/CMC)/CPC) for producing cultured meat.

### [Experimental Example 1] Evaluation of Properties of Crosslinked Porous Nanofilm

### 1.1 FT-IR Spectral Analysis of Crosslinked Porous Nanofilm (X-Linked (CHI/CMC))

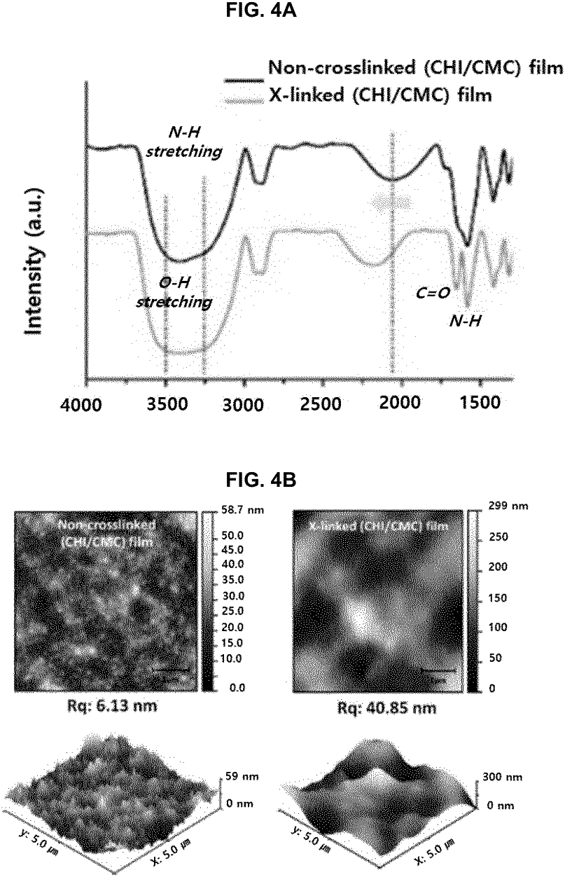

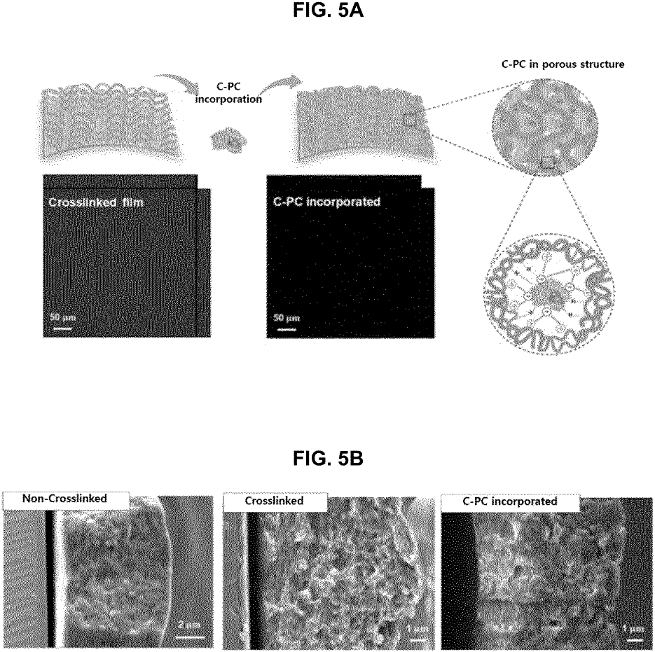

The qualitative analysis of the film before and after crosslinking and formation of additional bonds were investigated using Fourier transform infrared spectroscopy (FTIR, FT/IR-4700, Jasco, USA). The results are illustrated in FIG. 4A.

In the FT-IR spectrum of the (CHI/CMC) film, polysaccharide peaks corresponding to COC, COH, and CN were observed between 1,400 and 1,630 cm⁻¹, and overlapping peaks for OH and NH were observed between 3,200 to 3,500 cm⁻¹. In the case of the crosslinked film (X-linked (CHI/CMC)), the spectrum was similar to that of the non-crosslinked film (CHI/CMC), but O═C (1,680 cm⁻¹) and NH (1,645 cm⁻¹) bond peaks were additionally observed, and therefore, it could be confirmed that amide bonds were formed by crosslinking.

### 1-2. AFM Image Analysis of Crosslinked Porous Nanofilm (X-Linked (CHI/CMC))

The morphology of the film was observed by AFM and the image was analyzed using XEI and Gwyddion software. As illustrated in FIG. 4B, the (CHI/CMC) film showed a relatively dense morphology with an Rq value of 6.13 nm, whereas the crosslinked film (X-linked (CHI/CMC)) had an Rq value of 40.85 nm, which showed that a porosity was significantly increased.

### [Experimental Example 2] Evaluation of Nutrient Delivery Efficiency of Cell Culture Platform for Producing Cultured Meat

### Experiment for Release of C-Phycocyanin (C-PC)

The film sample produced according to Example 1 was coated on an OHP substrate, the substrate was applied to a cell culture plate, and then cultured murine C2C12 myoblasts (passage 10) were seeded in a 12-well plate at a concentration of 8 × 10³ cells/well. A culture medium containing 10% FBS and a culture medium containing 5% FBS were used as positive and negative control groups, respectively, and a culture medium containing 5% FBS was used for all groups using C-PC.

① A (CHI/CMC) film without C-PC, ② a (CHI/CMC)/CPC film group without a capping layer, ③ a (CHI/CMC)/CPC film with a capping layer, and ④ an exogenous C-PC group were used as experimental groups. The exogenous C-PC group was divided into two subgroups (Exo-CPC1 and Exo-CPC2), and in the case of Exo-CPC1, a medium containing C-PC was used in an amount equal to the total amount (93.22 µg/ml) of C-PC released from the capped film for 5 days. In the case of Exo-CPC2, a culture medium with daily addition of C-PC was used. At this time, the amount of C-PC added daily was calculated by dividing the total amount of C-PC released from the film by the number of days.

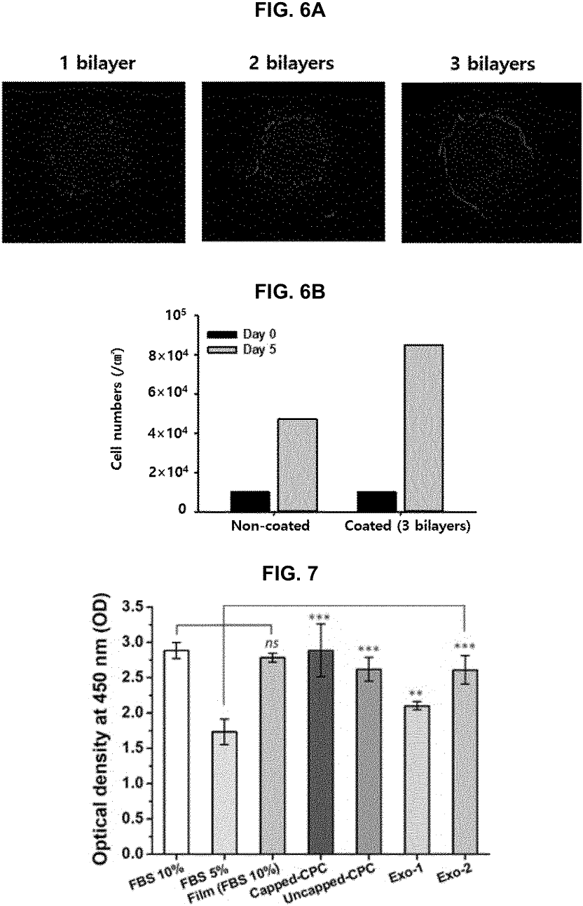

On the third day, the medium of the experimental group was replaced with a medium corresponding to each condition, and the film was not replaced. After the cells were incubated for a total of 5 days, the cells in the wells were washed with a 1X PBS buffer, and the results of the cell proliferation according to each experimental group were analyzed through CCK-8 assay. As illustrated in FIG. 6, all the groups containing C-PC showed a higher degree of cell proliferation than the FBS 5% group (negative control group). It is interpreted that C-PC had a positive effect on cell proliferation by supplementing the reduced FBS. A degree of cell proliferation in ③ the (CHI/CMC)/CPC film with a capping layer was almost similar to that of the FBS 10% group (positive control group) and was slightly higher than that of the Exo-CPC2 group, which was the highest among the experimental groups. The Exo-CPC1 group and the Exo-CPC2 group were finally treated with the same amount of C-PC, but a cell proliferation rate of the Exo-CPC2 group was significantly higher than that of the Exo-CPC1 group. These results are considered to be due to the higher activity of C-PC and the periodic stimulus applied to the cells by daily treatment of C-PC.

TABLE 1

| | Inital Initial | FBS 10% | FBS 5% | Uncapped | Capped film | Exo-1 | Exo-2 |
|---|---|---|---|---|---|---|---|
| Number of cells (x 10⁴ cells) | 0.8 | 19.63 + 1.61 | 3.2 + 2.6 | 15.85 ± 2.36 | 19.68 ± 5.34 | 8.44 ± 0.83 | 15.72 ± 2.86 |
| Expansion ratio | 1 | 24.53 | 3.99 | 19.81 | 24.60 | 10.55 | 19.63 |

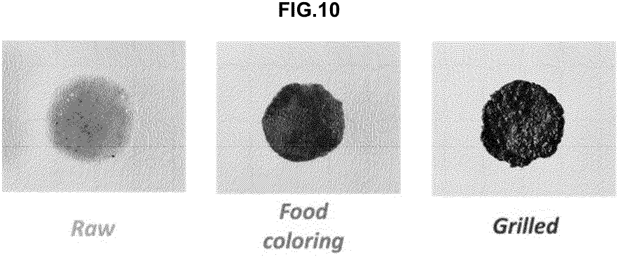

Table 1 shows the results of the number of cells and expansion ratio after culturing the cells for 5 days. In ③ the (CHI/CMC)/CPC film with a capping layer, it was observed that the cell proliferation was about 24 times higher than the number of initially seeded cells. As can be confirmed through the optical microscope images illustrated in FIG. 9, in the case of the negative control group and the Exo-CPC1 group, the cell density was relatively lower than that in other experimental groups, and it was observed that most of the cells were present in the form of unfused myoblasts. The other groups were saturated, and the fused root form was observed.

### [Example 1] Production of Cultured Meat Using Coated Stem Cells

Murine C2C12 myoblasts were prepared. A first coating (chitosan) solution at a concentration of 1 mg/ml was prepared by adding a chitosan aqueous solution (Sigma-Aldrich, USA) to DMEM (Thermo-Fischer, USA), and a second coating (CMC) solution at a concentration of 1 mg/ml was prepared by adding a carboxylmethylcellulose sodium salt aqueous solution (Sigma-Aldrich, USA) to DMEM. Myoblasts were immersed in 0.5 ml of the first coating solution for 1 minute to form a chitosan layer on surfaces of the myoblasts. Thereafter, the chitosan layer was immersed in 0.5 ml of the second coating solution for 1 minute to form a cell surface having a negatively charged CMC layer stacked on the chitosan layer. Myoblasts having surfaces coated with a chitosan and carboxymethylcellulose multilayer nanofilm were prepared by repeating the stacking 10 times based on electrostatic attraction, hydrogen bonding, and the biological interaction between chitosan and carboxymethylcellulose. DMEM in which chitosan and carboxymethylcellulose were not contained was used as a cleaning solution, and after forming a coating layer, the coating layer was immersed in the cleaning solution for 30 seconds to undergo a cleaning process. In addition, the replacement of the coating solution and the cleaning process were performed using a centrifuge.

The coated myoblasts were seeded in DMEM medium to which 5% FBS, 5% C-phycocyanin, and 1% Penicillin-Streptomycin (PS) were added, and the myoblasts were allowed to proliferate for 12 days, thereby forming myocytes. The proliferated cells were allowed to differentiate in DMEM differentiation medium to which 2% horse serum and 0.1% insulin were added for 7 days, and an electrical stimulus was applied at certain intervals to promote differentiation into muscle fibers. Subsequently, the muscle fibers were treated with beet root juice to color the muscle fibers to produce cultured meat.

### [Comparative Example 1] Production of Cultured Meat Using Uncoated Stem Cells

Cultured meat was produced in the same manner as that of Example 1 except for the process of coating murine myoblasts with chitosan and CMC.

However, when uncoated stem cells were used, a cell bundle was not sufficiently formed during long-term culture, and some cell death occurred due to external stimuli. It took more than 6 weeks to proliferate and differentiate into a large amount of myocytes for production of cultured meat, and it could be confirmed that a muscle fiber conversion was not properly performed.

### [Experimental Example 3] Evaluation of Cell Density and Differentiation After Proliferation According to Single Cell Coating

After proliferation of uncoated single cells (control group) and coated single cells using a trichrome staining method, the cell density and formed extracellular matrix were quantitatively analyzed. The effect of the coating on the cell proliferation may be evaluated by measuring an increase in weight of the coated single cells compared to the control group using a microbalance. The degree of differentiation in each of the control group and the coated single cells was evaluated by quantifying makers such as Pax7, MyoD, and Myogenin involved in proliferation and differentiation of myocytes using an immunoblotting technique.

The degree of proliferation of the single cells coated with the multilayer nanofilm was significantly increased compared to the control group, and as the cell proliferation was promoted, the time required for the single cells to reach confluency was much shorter. This is considered to be the result of enhanced adhesion between the cells through stabilization of cadherins involved in cell signal transduction by the multilayer nanofilm.

Although preferred embodiments of the present disclosure have been described, the present disclosure is not limited to the embodiments, but may be implemented in various modifications within the scope of the claims, the description of the invention, and the accompanying drawings. These modifications also fall within the scope of the present disclosure.

1. A method for producing cultured meat, comprising:
coating surfaces of cells usable for producing cultured meat to form a nanofilm;
culturing the coated cells;
inducing differentiation of the cultured cells; and
allowing muscle tissue to be formed from the differentiated cells.

2. The method of claim 1, wherein the cells usable for producing cultured meat are mesenchymal stem cells (MSCs), induced pluripotent stem cells (iPSCs), satellite cells, adipocytes, or embryonic stem cells.

3. The method of claim 1, wherein the coating is performed to form a multilayer nanofilm using one or two or more selected from the group consisting of electrostatic attraction, van der Waals force, hydrophobic bonding, hydrogen bonding, and covalent bonding.

4. The method of claim 1, wherein the nanofilm is formed by alternately stacking a positively charged material and a negatively charged material.

5. The method of claim 4, wherein the positively charged material and the negatively charged material form crosslinked complex.

6. The method of claim 4, wherein the positively charged material is one or two or more selected from the group consisting of chitosan, starch, collagen, gelatin, fibrinogen, silk fibroin, casein, elastin, laminin, and fibronectin.

7. The method of claim 4, wherein the negatively charged material is one or two or more selected from the group consisting of hyaluronic acid, alginate, pectin, tannic acid, lignin, cellulose, heparin, gellan gum, ester gum, carrageenan, agar, xanthan gum, gum arabic, glucomannan, carboxymethylcellulose gum (CMC), guar gum, locust bean gum, tamarind gum, and tara gum.

8. The method of claim 1, wherein a thickness of the nanofilm is 5 to 5,000 nm.

9. The method of claim 1, wherein the coated cells are cultured on a scaffold or in a bioreactor.

10. The method of claim 1, wherein in the culturing of the coated cells, the coated cells are stimulated by an ultrasonic wave, an electric current, an electromagnetic field, a magnetic field, or a combination thereof.

11. The method of claim 1, further comprising adding a fat and a colorant to the muscle tissue.

12. Cultured meat produced by the method for producing cultured meat of claim 1.

13. The cultured meat of claim 12, wherein the cultured meat is a substitute for chicken, pork, beef, goat, lamb, duck, or fish.
